(19) United States
(12) Patent Application Publication
(10) Pub. No.: US 2008/0268039 A1
(43) Pub. Date: Oct. 30, 2008

(54) LOQUAT COMPOSITIONS

(75) Inventor: Michael Fantuzzi, Glendale, CA (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(73) Assignee: Soft Gel Technologies, Inc., Los Angeles, CA (US)

(21) Appl. No.: 12/042,613

(22) Filed: Mar. 5, 2008

Related U.S. Application Data
(60) Provisional application No. 60/893,307, filed on Mar. 6, 2007.
(60) Provisional application No. 60/893,497, filed on Mar. 7, 2007.

(51) Int. Cl. A61K 9/48; A61P 3/08

(52) U.S. Cl. 424/456

(57) ABSTRACT

Soft gelatin capsules containing a Loquat extract are described herein having increased bioavailability and efficacy over known materials.

### CROSS-REFERENCE TO RELATED APPLICATION(S)

This application claims benefit under 35 U.S.C. § 119(e) to U.S. Ser. No. 60/893,307, entitled “Loquat Compositions”, filed Mar. 6, 2007 (attorney docket number 189079/US) by Michael Fantuzzi and U.S. Ser. No. 60/893,497, entitled “Loquat Compositions”, filed Mar. 7, 2007 (attorney docket number 189079/US/2) by Michael Fantuzzi, the contents of which are incorporated herein by reference in their entirety.

### FIELD OF THE INVENTION

The present invention relates to a corosolic extract from Loquat that is encapsulated in a soft gelatin capsule.

### BACKGROUND OF THE INVENTION

The first diagnosis of diabetes dates back to Greece, 2,000 years ago. Blood sugar balance, in general, diabetes, in particular, ever since has been the subject of an increasing scientific study. Diabetes affects 16 million people in the United States alone and it is the fourth leading cause of death. Insulin, the hormone produced by pancreas, regulates the uptake and conversion of sugar into heat energy and muscle power. Diabetes is a metabolic disorder and insufficient insulin production leads to Type 1 diabetes or insulin-dependent diabetes mellitus (IDDM). Lipid metabolism is often deranged in diabetics resulting in weight gain and other complications.

More than half of U.S. adults are overweight (body mass index, BMI<25), one-quarter is obese (BMI<30), and 11% of children and adolescents are overweight. Approximately 280,000 deaths are attributable to obesity annually. Sedentary life style is prevalent and only 22% of U.S. adults exercise the recommended five times per week for at least 30 minutes. Healthy weight maintenance involves a delicate balance between energy intake and energy expenditure.

Glucose is the principal nutrient for energy and daily energy balance between intake and expenditure is a determining factor in body weight stability. A long-term positive energy balance leads to weight gain, while a negative balance accounts for weight loss. Obesity is an alarming trend globally and more acute in developed countries due to sedentary life style and rich diets among both adults and children and leads to deleterious consequences such as obesity, syndrome X, insulin resistance, diabetes and other health risks. Syndrome X is a metabolic disorder characterized by insulin resistance and central obesity, high cholesterol, high blood pressure and high blood sugar levels. An estimated 20 to 30% of middle-aged Americans suffer from Syndrome X, which is believed to increase risk for diabetes and heart disease. The spread of obesity is considered to be an epidemic in the U.S. and a sensible, sustained weight management is a critical step in this environment.

Glucose is an important nutrient for many cells of the body. Glucose transport from the blood into cells, therefore, is an important function of all cells and some tissues, such as brain, are solely dependent on glucose as an energy source. Insulin regulates glucose uptake into fat and muscle cells through the recruitment of glucose transporter (GLUT)4 from an intracellular membrane storage pool to the plasma membrane. A complex homeostatic mechanism keeps the blood glucose level constant in mammals and most cells contain several types of sodium linked glucose transporters known as GLUT family. Glucose transporters, such as GLUT4, are especially important for regulating intracellular glucose in heart and skeletal muscle cells and in fat cells (brown and white adipocytes). The pancreatic hormone insulin regulates blood sugar levels by a cascade of biochemical steps, including activation and translocation of GLUT4 to cell surface, for glucose transport from blood to cells.

Numerous groups have been systematically searching for an agent to modify glucose transport activity and to find a natural product useful as an anti-diabetic agent. Various medicinal plants from Asia have been used to treat diabetes and the plants exhibiting hypoglycemic effect include Momordica Charantia, Tinospora Cordifolia, Ginseng, etc. Tea preparations from the leaves of Lagerstroemia Speciosa L., traditionally have been used for weight-loss and by diabetics to balance blood sugar levels and in-vitro studies indicate that corosolic acid extracted from the leaves of Lagerstroemia Speciosa L, improves the cellular uptake of glucose. Further studies in diabetic mice indicate the hypoglycemic effects of leaf-extracts from Lagerstroemia Speciosa L.

However, Lagerstroemia Speciosa L is expensive in terms of harvesting, growing and processing. Other suitable plants sources that produce corosolic acid are available but have not been utilized until this time.

Therefore, compositions and methods for treating and preventing blood glucose metabolism disorders continue to be sought through new research and development of corosolic acid sources.

### BRIEF SUMMARY OF THE INVENTION

The present invention pertains to the surprising discovery that a new corosolic acid containing extract from Loquat provides a increased amounts of bioavailable components of the extract, including corosolic acid, via delivery in a soft gelatin capsule. The Loquat extract contained within the soft gelatin capsule provides compositions that help to maintain or lower blood sugar levels and/or lower body fat in an individual in need thereof

The compositions of the invention are useful as dietary supplements or as nutriceuticals.

In particular, the compositions of the invention are included in a soft gelatin (soft gel) capsule. Typically, the soft gelatin capsule includes at least 5% by weight of the Loquat extract. Typically the Loquat extract has a corosolic acid concentration of at least 1 percent, more particularly 3 percent, even more particularly greater than 10 percent, more particularly at least 18 percent and up to 95 percent by weight of dried extract. Additionally, the components of the extract, although not identified, have a surprising synergistic effect on the bioavailability and efficacy when delivered to an individual in need thereof. Not to be limited by theory, it is believed that the extraction process helps to concentrate and possible reduce or eliminate degradation of essential components that would otherwise not be stable when isolated by other methods.

In another embodiment, the present invention pertains to methods for delivery of an effective amount of bioavailable Loquat extract, containing corosolic acid, such that an effective amount of corosolic acid and/or other active components of the Loquat extract is (are) provided to the individual.

In still another embodiment, the present invention also includes packaged formulations of the invention and instructions for use of the soft gelatin capsule that encapsulates the Loquat extract.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### DETAILED DESCRIPTION

The present invention pertains to the surprising discovery that extracts of Loquat provide corosolic acid as well as other beneficial active components that help to maintain or reduce blood sugar levels and/or body weight reduction in individuals in need thereof. Therefore, the present invention provides Loquat extract compositions encapsulated with in soft gel formulations that are suitable for inhibiting an increase in, or lowering, a blood sugar level in an individual. Additionally, the soft gelatin containing Loquat extract is useful for maintenance of and or reduction of moderate weight loss through blood sugar maintenance.

The soft gelatin containing Loquat extract is useful also to treat obesity, syndrome X, insulin resistance, diabetes and other health risks. Syndrome X is a metabolic disorder characterized by insulin resistance and central obesity, high cholesterol, high blood pressure and high blood sugar levels. Thus, the compositions of the invention can be used to treat this conditions.

The product relies on the effects of corosolic acid, as well as other yet unidentified active components of the Loquat extract, on blood sugar levels to derive a healthy weight loss effect for Type II diabetics (non-insulin dependent) and healthy non-diabetics and the improved absorption of an oil based delivery system. The product provides safe and sustainable weight loss when combined with a restricted calorie diet and regular exercise. Its benefits include improvement of cardiovascular health, normalized blood sugar levels, and improved physical appearance with the positive psychological effects associated with successful and safe weight loss/maintenance.

In one embodiment, a soft gel formulation includes the Loquat extract including corosolic acid, rice bran oil, and/or yellow bee's wax and/or silica and/or other additives or carriers. In one aspect, the soft gel Loquat formulation is administered thrice a day in dosages of corosolic acid of about 0.5 to about 100 mg per capsule, in particular about 10 to about 50 mg per capsule, and in more particular, about 15 to about 25 mg per capsule. This understanding should not be limited to just corosolic acid; the weight of the extract includes other active agents that are helpful in therapeutic treatments as identified herein.

In the specification and in the claims, the terms “including” and “comprising” are open-ended terms and should be interpreted to mean “including, but not limited to. . . . ” These terms encompass the more restrictive terms “consisting essentially of” and “consisting of.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The phrase “Loquat extract” is intended to encompass both liquid and solid (e.g., powdered) isolated materials from Loquat. As used herein, “Loquat” refers to the plant species Eriobotrya japonica, (Thunb.) Lindl. of the family rosaceae, also known by various common names such as Rosaceae Advance, Champagne, Early Red, Japanese medlar, Pi Pa Ye, Japanese plum, Nispero, etc., including all well known strains, variations, and hybrids thereof, grown anywhere in the world.

The term “extract” when used in connection with a plant such as “Loquat”, refers to one or more active agents, or a composition containing such, that is obtained from the plant, or a portion thereof, including the flower, fruit, seed, peel, leaf, root, and bark. As will be recognized by those of ordinary skill in the art, extracts may be either crude or refined to a selected degree in order to isolate specified active agents. A number of extraction processes that can be employed to produce the compositions of various types will be recognized by those of ordinary skill in the art.

The extraction process of the present invention includes the following steps. After the Loquat source material has been obtained, it is partitioned into smaller pieces. The small pieces are then soaked in an alcoholic or alcoholic/aqueous solution for about 1 to about 4 hours to form an extract solution. The extract solution is filtered, and the filtrate containing the target active agents is collected and concentrated. Following concentration, the filtrate may be dried, such as by freeze-drying, in order to provide an extract having a solid form.

Suitable alcohols include ethanol, methanol, cellosolve, propanol, butanol, and lower molecular weight alcohols and polyols.

Various soaking conditions may be used in the course of the present extraction method, in order to achieve a specific final Loquat extract. In one aspect of the present invention, the aqueous solution may include distilled water, and from about 1% to about 95% alcohol. In another aspect, the alcohol concentration may be greater than about 50%. In yet another aspect, the alcohol concentration may be greater than about 80%. In an additional aspect, the alcohol concentration may be greater than about 90%. In one aspect of the invention, the temperature of the aqueous solution may be from about room temperature to about 95° C. In another aspect, the temperature may be from about 50° C. to about 70° C. As noted above, the amount of time for which the Loquat pieces are soaked may be from about 1 to about 48 hours. However, in one aspect, the amount of time may be about 24 to about 48 hours. In another aspect, the soaking time is at an elevated temperature, over the 24 to 48 hour period of a range of about 30 to about 90° C. It has been found unexpectedly that the extended periods of soak help to increase the corosolic acid and active ingredient components in the final extract and provide enhanced bioavailability and efficacy.

After the Loquat pieces have been sufficiently soaked, the resultant extract solution is filtered to separate the pieces from the liquid filtrate containing the desirable bioactive agents. Filtering may be accomplished using a number of specific filtration processes known to those of ordinary skill in the art, such as reduced pressure vacuum filtration, and may employ a number of different suitable filter types, such as paper filters, etc.

The filtrate that contains the desired bioactive agents such as corosolic acid, is collected. The volatile solvent(s) are then removed by any suitable method known to those of ordinary skill in the art, such as distillation, filtration, separation reaction, etc. The remaining extract is then condensed or concentrated. In some aspects, the concentrated extract may further be dried, such as by freeze-drying, or other techniques known to those of ordinary skill in the art, in order to provide a solid or powdered form extract. Such an extract may then be combined with other ingredients to provide a suitable dosage form for administration as discussed in further detail below.

In one aspect, the filtrate is filtered over a polymeric resin such as Amberlite or similar resin. Adsorption of unknown materials apparently helps to provide a more active extract having enhanced efficacy and bioavailability when compared to materials not treated in such fashion.

In one aspect of the present invention the extraction process may yield an extract that is standardized to contain 18 or greater % w/w of the active ingredient corosolic acid. This standardized content allows various dosage formulations to be made which contain a target corosolic acid dosage. While a variety of amounts may be used, in one aspect, the composition may be formulated to contain from about 0.5% w/w to about 40% w/w corosolic acid, more particularly from about 1% to about 35% w/w, more particularly from about 3% to about 25% w/w and even more particularly from about 5% to about 18% w/w. Other bioactive agents that are thought to also play a role in facilitating glucose metabolism, such as various sesqiterpenoids, are also contained in the Loquat extract obtained by the extraction process of the present invention.

A useful commercial source of Loquat extract is available from Tokiwa Phytochemical Co., Ltd., Japan (Loquat leaf extract).

Formulation of the Loquat extract can include, optionally, additional components, and can be accomplished by many methods known in the art. For example, the compositions of the invention can be formulated in as a unitary liquid, a suspension, an emulsion and encapsulated in a soft gelatin capsule (that harbors the liquid). Often the formulation will include an acceptable carrier, such as an oil, or other suspending agent.

Suitable carriers include but are not limited to, for example, fatty acids, esters and salts thereof, that can be derived from any source, including, without limitation, natural or synthetic oils, fats, waxes or combinations thereof. Moreover, the fatty acids can be derived, without limitation, from non-hydrogenated oils, partially hydrogenated oils, fully hydrogenated oils or combinations thereof. Non-limiting exemplary sources of fatty acids (their esters and salts) include seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba, tallow, beef tallow, butter, chicken fat, lard, dairy butterfat, shea butter or combinations thereof.

Specific non-limiting exemplary fish or marine oil sources include shellfish oil, tuna oil, mackerel oil, salmon oil, menhaden, anchovy, herring, trout, sardines or combinations thereof. In particular, the source of the fatty acids is fish or marine oil (DHA or EPA), soybean oil or flaxseed oil. Alternatively or in combination with one of the above identified carriers, beeswax can be used as a suitable carrier, as well as suspending agents such as silica (silicon dioxide).

Alternatively, the formulations of the invention are also considered to be nutraceuticals. The term “nutraceutical” is recognized in the art and is intended to describe specific chemical compounds found in foods that may treat or prevent disease conditions.

Disease conditions include those noted throughout the application including, glucose metabolism disorders, such as diabetes mellitus, insulin resistance, hyperglycemia, and hyperinsulinemia.

The formulations of the invention can further include various ingredients to help stabilize, or help promote the bioavailability of the Loquat extract and active ingredients thereof or serve as additional nutrients to an individual's diet. Suitable additives can include vitamins and biologically-acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals may be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Vitamin(s), if present, are present in the composition of the invention in an amount ranging from about 5 mg to about 500 mg. More particularly, the vitamin(s) is present in an amount ranging from about 10 mg to about 400 mg. Even more specifically, the vitamin(s) is present from about 250 mg to about 400 mg. Most specifically, the vitamin(s) is present in an amount ranging from about 10 mg to about 50 mg. For example, B vitamins are in usually incorporated in the range of about 1 milligram to about 10 milligrams, i.e., from about 3 micrograms to about 50 micrograms of B12. Folic acid, for example, is generally incorporated in a range of about 50 to about 400 micrograms, biotin is generally incorporated in a range of about 25 to about 700 micrograms and cyanocobalamin is incorporated in a range of about 3 micrograms to about 50 micrograms.

Mineral(s), if present, are present in the composition of the invention in an amount ranging from about 25 mg to about 1000 mg. More particularly, the mineral(s) are present in the composition ranging from about 25 mg to about 500 mg. Even more particularly, the mineral(s) are present in the composition in an amount ranging from about 100 mg to about 600 mg.

Various additives can be incorporated into the present compositions. Optional additives of the present composition include, without limitation, phospholipids, L-carnitine, starches, sugars, fats, antioxidants, amino acids, proteins, flavorings, coloring agents, hydrolyzed starch(es) and derivatives thereof or combinations thereof.

As used herein, the term “phospholipid” is recognized in the art, and refers to phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins.

As used herein, the term “antioxidant” is recognized in the art and refers to synthetic or natural substances that prevent or delay the oxidative deterioration of a compound. Exemplary antioxidants include tocopherols, flavonoids, catechins, superoxide dismutase, lecithin, gamma oryzanol; vitamins, such as vitamins A, C (ascorbic acid) and E and beta-carotene; natural components such as camosol, carnosic acid and rosmanol found in rosemary and hawthorn extract, proanthocyanidins such as those found in grapeseed or pine bark extract, and green tea extract.

The term “flavonoid” as used herein is recognized in the art and is intended to include those plant pigments found in many foods that are thought to help protect the body from cancer. These include, for example, epi-gallo catechin gallate (EGCG), epi-gallo catechin (EGC) and epi-catechin (EC).

Any dosage form, and combinations thereof, are contemplated by the present invention. Examples of such dosage forms include, without limitation, chewable tablets, elixirs, liquids, solutions, suspensions, emulsions, capsules, soft gelatin capsules, hard gelatin capsules, caplets, lozenges, chewable lozenges, suppositories, creams, topicals, ingestibles, injectables, infusions, health bars, confections, animal feeds, cereals, cereal coatings, and combinations thereof. The preparation of the above dosage forms are well known to persons of ordinary skill in the art.

Soft gel or soft gelatin capsules can be prepared, for example, without limitation, by dispersing the formulation in an appropriate vehicle (e.g. rice bran oil, monoterpene and/or beeswax) to form a high viscosity mixture. This solution is then encapsulated with a gelatin based film using technology and machinery known to those in the soft gel industry. The industrial units so formed are then dried to constant weight. Typically, the weight of the capsule is between about 100 to about 2500 milligrams and in particular weigh between about 1500 and about 1900 milligrams, and more specifically can weigh between about 1500 and about 2000 milligrams.

For example, when preparing soft gelatin shells, the shell can include between about 20 to 70 percent gelatin, generally a plasticizer and about 5 to about 60% by weight sorbitol. The filling of the soft gelatin capsule is liquid (principally rice bran oil and/or beeswax if desired) and can include, apart form the antioxidant actives, a hydrophilic matrix. The hydrophilic matrix, if present, is a polyethylene glycol having an average molecular weight of from about 200 to 1000. Further ingredients are optionally thickening agents. In one embodiment, the hydrophilic matrix includes polyethylene glycol having an average molecular weight of from about 200 to 1000, 5 to 15% glycerol, and 5 to 15% by weight of water. The polyethylene glycol can also be mixed with propylene glycol and/or propylene carbonate.

In another embodiment, the soft gel capsule is prepared from gelatin, glycerine, water and various additives. Typically, the percentage (by weight) of the gelatin is between about 30 and about 50 weight percent, in particular between about 35 and about weight percent and more specifically about 42 weight percent. The formulation includes between about 15 and about 25 weight percent glycerine, more particularly between about 17 and about 23 weight percent and more specifically about 20 weight percent glycerine.

The remaining portion of the capsule is typically water. The amount varies from between about 25 weigh percent and about 40 weight percent, more particularly between about 30 and about 35 weight percent, and more specifically about 35 weight percent. The remainder of the capsule can vary, generally, between about 2 and about 10 weight percent composed of a flavoring agent(s), sugar, coloring agent(s), etc. or combination thereof. After the capsule is processed, the water content of the final capsule is often between about 5 and about 10 weight percent, more particularly 7 and about 12 weight percent, and more specifically between about 9 and about 10 weight percent.

As for the manufacturing , it is contemplated that standard soft shell gelatin capsule manufacturing techniques can be used to prepare the soft-shell product. Examples of useful manufacturing techniques are the plate process, the rotary die process pioneered by R. P. Scherer, the process using the Norton capsule machine, and the Accogel machine and process developed by Lederle. Each of these processes are mature technologies and are all widely available to any one wishing to prepare soft gelatin capsules.

Typically, when a soft gel capsule is prepared, the total weight is between about 250 milligrams and about 2.5 gram in weight, e.g., 400-750 milligrams. Therefore, the total weight of additives, such as vitamins and antioxidants, is between about 80 milligrams and about 2000 milligrams, alternatively, between about 100 milligrams and about 1500 milligrams, and in particular between about 120 milligrams and about 1200 milligrams. In particular, the soft gel capsule typically weighs between about 1000 milligrams and 1300 milligrams, wherein the percentage fill is about 50% of the entire weight of the capsule, i.e., from about 500 to about 650 milligrams fill weight. The fill weight includes the active ingredient(s), solubilizing agents, etc.

Preparation of the soft gel capsules was accomplished by methods well known in the art including, but not limited to those described throughout the specification and in U.S. Pat. Nos. 6,616,942, 6,623,734 and pending U.S. Ser. Nos. 10/035,753 and 09/825,920, the contents of which are incorporated herein by reference in their entirety.

For example, a soft gel capsule can be prepared by mixing the Loquat extract with a carrier. The mixture is then combined with encapsulated within a gelatin capsule as described above.

For example, rice bran oil is poured into a blending machine, and optionally, yellow beeswax is added to it. The mixture is then warmed until the components are melted and mixed until a homogeneous solution is obtained.

The solution is cooled to about room temperature and thickening and or suspending agent can be added, such as fume silica. Loquat extract can then be added and the mixture is stirred, generally under vacuum and an elevated temperature to keep the mixture liquified for a period of time to ensure homogeniety of the mixture.

The mixture can then be encapsulated.

Selection of Loquat extracts with standardized amounts of corosolic acid can be used to prepare soft gelatin capsules having varying amounts of active components.

The present invention also provides packaged formulations and instructions for use of the capsule. Typically, the packaged formulation, in whatever form, is administered to an individual in need thereof that requires an increase in the amount of corosolic acid in the individual's diet. Typically, the dosage requirement is between about 1 to about 4 dosages a day.

The following paragraphs enumerated consecutively from 1 through 15 provide for various aspects of the present invention. In one embodiment, in a first paragraph (1), the present invention provides a soft gelatin capsule, comprising a composition that includes a Loquat extract having at least about 10% corosolic acid content of the weight of the extract; and a carrier.

2. The soft gelatin capsule of paragraph 1, wherein the soft gelatin tablet further comprises rice bran oil or beeswax.

3. The soft gelatin capsule of any of paragraphs 1 or 2, further comprising vitamin E.

4. The soft gelatin capsule of any of paragraphs 1 through 3, further comprising a seed oil.

5. The soft gelatin capsule of any of paragraphs 1 through 4, further comprising a fish oil

6. A method to lower or maintain blood glucose levels comprising the step of administering to an individual in need thereof a soft gelatin capsule comprising a composition comprising a Loquat extract having at least about 10% corosolic acid content; and a carrier.

7. The soft gelatin capsule of any of paragraphs 1 through 5 or method of paragraph 6, wherein the corosolic acid content is about 18% by weight of the extract.

8. The soft gelatin capsule of paragraph 7 or method of paragraph 7, wherein the corosolic acid content is about 25% of the extract.

9. A soft gelatin capsule, comprising a composition comprising a Loquat extract that includes corosolic acid; and a carrier, such that the composition is encapsulated within the soft gelatin capsule.

10. The soft gelatin capsule of paragraph 9, wherein the carrier is rice bran oil.

11. The soft gelatin capsule of paragraph 10, further comprising beeswax or lecithin.

12. The soft gelatin capsule of paragraph 11, further comprising a thickening agent.

13. The soft gelatin capsule of paragraph 12, wherein the thickening agent is fumed silica.

14. A method to lower or maintain blood glucose levels comprising the step of administering to an individual in need thereof a soft gelatin capsule comprising a composition comprising a Loquat extract that includes corosolic acid; and a carrier, such that the composition is encapsulated within the soft gelatin capsule, wherein the individual's blood glucose level is lowered or maintained.

15. A method for weight loss comprising the step of administering to an individual in need thereof a soft gelatin capsule comprising a composition comprising a Loquat extract that includes corosolic acid; and a carrier, such that the composition is encapsulated within the soft gelatin capsule, wherein the individual's weight is reduced.

The following examples are not to be meant as limiting but are presented to provide additional information and support for the invention.

### EXAMPLES

The following is a general process used to prepare soft gelatin capsules containing varying amounts of Loquat extract and corosolic acid content.

Rice bran oil was added to a blending machine and yellow beeswax was added to it. The mixture was completely melted and stirred at 60° C.

The mixture was allowed to cool to at least 30° C. Fumed silica or lecithin and Loquat extract (containing a standardized amount of corosolic acid) (Tokiwa Phytochemical Co., Ltd., Japan) were then added. The resulting mixture was stirred under vacuum at 30° C. for at least 30 minutes.

The resultant mixture (a liquid) was checked for large particles, and if there were less than 5% of the material that had particle sizes less than 6/1000ths of an inch, the material was sent to be encapsulated. If there was more than 5% of the material that had particle sizes larger than 6/1000ths of an inch, then the material was passed through a stone mill to reduce the particle sizes to within the specification set above, then de-aerated by vacuum and then encapsulated.

| | |
|---|---|
| 1% Corosolic acid (0.48 mg): | |
| 1% Corosolic acid content | 48.00 mg |
| Fumed Silica | 8.00 mg |
| Yellow Beeswax | 15.00 mg |
| Rice Bran Oil | 359.00 mg |
| Total capsule fill weight | 430.00 mg |
| 3% Corosolic acid (0.72 mg): | |
| 3% Corosolic acid content | 24.00 mg |
| Fumed Silica | 8.00 mg |
| Yellow Beeswax | 19.00 mg |
| Rice Bran Oil | 379.00 mg |
| Total capsule fill weight | 430.00 mg |
| 18% Corosolic acid (0.48 mg): | |
| 18% Corosolic acid content | 2.667 mg |
| Fumed Silica | 8.000 mg |
| Yellow Beeswax | 19.00 mg |
| Rice Bran Oil | 400.333 mg |
| Total capsule fill weight | 430.00 mg |
| 18% Corosolic acid (0.72 mg): | |
| 18% Corosolic acid content | 4.00 mg |
| Fumed Silica | 8.00 mg |
| Yellow Beeswax | 21.00 mg |
| Rice Bran Oil | 397.00 mg |
| Total capsule fill weight | 430.00 mg |
| 18% Corosolic acid (0.10 mg): | |
| 18% Corosolic acid content | 56.00 mg |
| Yellow Beeswax | 26.00 mg |
| Lecithin | 8.00 mg |
| Rice Bran Oil | 210.00 mg |
| Total capsule fill weight | 300.00 mg |

Although the present invention has been described with reference to preferred embodiments, persons skilled in the art will recognize that changes may be made in form and detail without departing from the spirit and scope of the invention.

All literature and patent references cited throughout the application are incorporated by reference into the application for all purposes.

What is claimed is:

1. A soft gelatin capsule, comprising a composition comprising:
a Loquat extract that includes corosolic acid; and
a carrier, such that the composition is encapsulated within the soft gelatin capsule.

2. The soft gelatin capsule of claim 1, wherein the carrier is rice bran oil.

3. The soft gelatin capsule of claim 2, further comprising beeswax or lecithin.

4. The soft gelatin capsule of claim 3, further comprising a thickening agent.

5. The soft gelatin capsule of claim 4, wherein the thickening agent is fumed silica.

6. A method to lower or maintain blood glucose levels comprising the step of administering to an individual in need thereof a soft gelatin capsule comprising a composition comprising:
a Loquat extract that includes corosolic acid; and
a carrier, such that the composition is encapsulated within the soft gelatin capsule, wherein the individual's blood glucose level is lowered or maintained.

7. A method for weight loss comprising the step of administering to an individual in need thereof a soft gelatin capsule comprising a composition comprising: a Loquat extract that includes corosolic acid; and
a carrier, such that the composition is encapsulated within the soft gelatin capsule, wherein the individual's weight is reduced.
